(19) United States
(12) Patent Application Publication
(10) Pub. No.: US 2023/0391825 A1
(43) Pub. Date: Dec. 7, 2023

(54) ENHANCED SKIN PERMEATION OF A NOVEL PEPTIDE VIA STRUCTURAL MODIFICATION, CHEMICAL ENHANCEMENT, AND MICRONEEDLES

(71) Applicant: ESCAPE THERAPEUTICS, INC., Turlock, CA (US)

(72) Inventor: Basil M. Hantash, Turlock, CA (US)

(21) Appl. No.: 18/249,521

(22) PCT Filed: Oct. 19, 2021
(86) PCT No.: PCT/US2021/055692

§ 371 Date: Apr. 18, 2023

Related U.S. Application Data
(60) Provisional application No. 63/094,242, filed on Oct. 20, 2020.

(51) Int. Cl. C07K 7/06; A61K 8/36; A61K 8/35; A61K 8/34; A61K 9/00; A61Q 19/02; A61K 8/64

(52) U.S. Cl. CPC C07K 7/06; A61K 8/361; A61K 8/35; A61K 8/34; A61K 9/0021; A61K 9/0014; A61Q 19/02; A61K 8/345; A61K 8/64

(57) ABSTRACT

Hyperpigmentation is a common skin condition with serious psychosocial consequences. Decapeptide-12, a newly synthesized peptide, has been found to be safer than hydroquinone in reducing content of melanin, with efficacy up to more than 50 percent upon 16 weeks of twice daily treatment. However, the peptide suffers from limited transcutaneous penetration due to its hydrophilicity and large molecular weight. Therefore, decapeptide-12 was modified by adding a palmitate chain in an attempt to overcome this limitation. We also tested the effects of chemical penetration enhancers and microneedles to deliver two peptides through skin. Enhanced human skin permeation was found using an in vitro skin permeation model. Moreover, we examined peptide retention of different formulations. Our data showed that palm-peptides in microneedle patch was the most effective.

### CROSS-REFERENCE TO RELATED APPLICATION

The instant nonprovisional patent application claims priority to U.S. patent application 63/094,242, filed Oct. 20, 2020, which is incorporated by reference along with all other references cited in this application.

### SEQUENCE LISTING

None.

### BACKGROUND OF THE INVENTION

This invention relates to the field of novel biological agents.

### BRIEF SUMMARY OF THE INVENTION

Hyperpigmentation is an example of common skin condition with serious psychosocial consequences. Decapeptide-12, a newly synthesized peptide, has been found to be safer than hydroquinone in reducing content of melanin, with efficacy up to more than 50 percent upon 16 weeks of twice daily treatment. However, the peptide suffers from limited transcutaneous penetration due to its hydrophilicity and large molecular weight. Therefore, decapeptide-12 was modified by adding a palmitate chain in an attempt to overcome this limitation. We also tested the effects of chemical penetration enhancers and microneedles to deliver two peptides through skin. Enhanced human skin permeation was found using an in vitro skin permeation model. Moreover, we examined peptide retention of different formulations. Our data showed that palm-peptides in microneedle patch was the most effective.

Hyperpigmentation and melasma are merely two examples of a condition that can be treated. Other conditions can be treated including: neurological, dermatological, oncological, immunological, and others. The peptide can treat internal and skin conditions, not only melasma or hyperpigmentation. This peptide has anti-inflammatory and anti-senescence functions which impact every system of the body and every organ of the body. Thus, this peptide can cover all conditions impacting the body and its organs and systems.

There are two approaches to organs and systems noted below. Further anti-aging does not require treatment of something that is considered a disease or abnormal. We also are able to treat something normal that someone wants to make better or improve it.

There are eleven organ systems including the integumentary system, skeletal system, muscular system, lymphatic system, respiratory system, digestive system, nervous system, endocrine system, cardiovascular system, urinary system, and reproductive systems, which this peptide can treat or treat conditions of.

There are fourteen disease systems including: the Musculoskeletal system; Organs of Special Sense (optical); Auditory; Infectious Diseases, Immune Disorders, and Nutritional Deficiencies; Respiratory system; Cardiovascular system; Digestive system; Genitourinary System; Hemic and Lymphatic system; Skin; Endocrine system; Neurological conditions; Mental Disorders, and Dental and Oral conditions. The peptide can be used to treat these diseases and systems.

Other objects, features, and advantages of the present invention will become apparent upon consideration of the following detailed description and the accompanying drawings, in which like reference designations represent like features throughout the figures.

### BRIEF DESCRIPTION OF THE DRAWINGS

FIGS. 1a and 1b show structures of peptides. Molecular structures of the two peptides: native peptide (1a) and its analogue, palm-peptide (1b). For palm-peptide, N-terminal was palmitoylated, C-terminal was modified to amide, and tyrosine at position 6 was changed from L- to D-.

FIG. 2 shows a flow for a synthesis of the peptides.

FIGS. 3a and 3b show a solubility of two peptides in three enhancers in PG. Group1: Native peptide; Group2: Native peptide+enhancer1; Group3: Native peptide+enhancer2; Group4: Native peptide+enhancer3; Group5: Palm-D-ISO-Amid; Group6: Palm-D-ISO-Amid+enhancer1; Group7: Palm-D-ISO-Amid+enhancer2; Group8: Palm-D-ISO-Amid+enhancer3. Cumulative permeation profiles of the two different peptides over 24 hours in different drug delivery systems. Native peptide in MN patch and palm-peptide in PG, PG with 5 percent (weight/volune) oleic acid, PG with 5 percent (weight/volume) camphor, PG with 5 percent (weight/volume) menthol, and MN patch: amounts (a) and percentage (b). MN=microneedle, PG=propylene glycol.

FIGS. 4a and 4b show a cumulative skin permeation of two peptides in three enhancers in PG over 24 hours, in amounts (4a) and percentage (4b).

FIGS. 5a and 5b show cumulative amount of peptide permeated through skin after 24 hours in amounts (a) and percentage (b).

FIGS. 6a and 6b show a skin retention of two peptides after 24 hours, in amounts (6a) and percentage (6b). Retention of native peptide in MN patch, palm-peptide in 5 percent (weight/volume) camphor/menthol were not detected.

### DETAILED DESCRIPTION OF THE INVENTION

Melanin, the end product of melanogenesis, plays a crucial role in the absorption of free radicals generated within the cytoplasm, in shielding the host from various types of ionizing radiations, and determining the color of human skin, hair, and eyes. However, excess production of melanin can cause skin hyperpigmentation, a common and non-life-threatening disorder. Melasma is a form of hyperpigmentation that causes brown or gray patches on skin, primarily in the facial area. Hydroquinone and tretinoin, in combination with topical corticosteroids are well established therapeutic agents for melasma and hyperpigmentation treatment. Hantash and his group reported that a novel proprietary synthetic peptide, namely, decapeptide-12, which demonstrated a stronger competitive inhibition effect on both mushroom and human tyrosinase enzymes than hydroquinone. Additional studies on humans demonstrated the therapeutic effect of Lumixyl™, a topical formulation containing decapeptide-12, for the treatment of melasma.

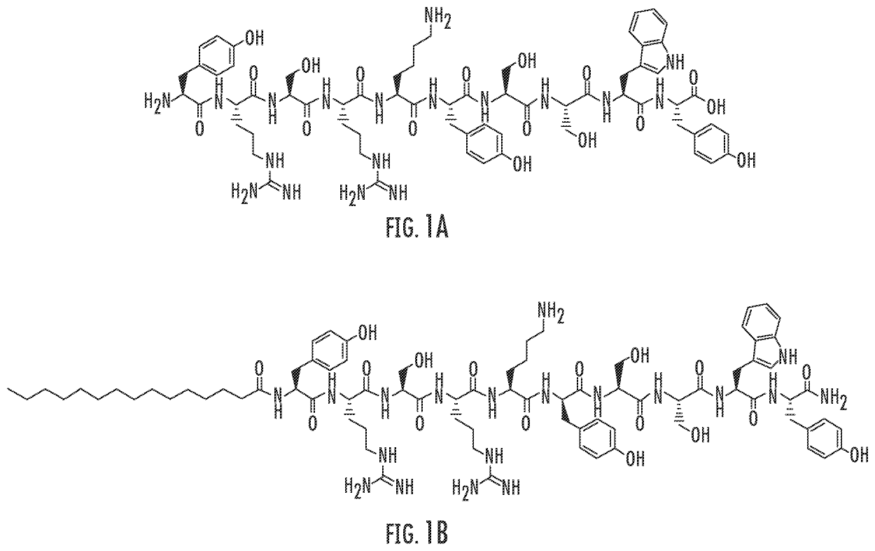

While decapeptide-12 could reduce production of melanin with good efficacy in treating melasma, the permeation profile of decapeptide-12 remains unclear. As shown in FIG. 1a, decapeptide-12 is a polar molecule with multiple amino and hydroxyl groups, which may hinder its transdermal absorption through the lipophilic stratum corneum (SC). To this end, multiple options are available, e.g., molecular modification, using chemical penetration enhancers (CPEs) or microneedles (MNs).

For molecular modification, many studies are focused on improving the biostability and bioavailability of peptides for oral, pulmonary, and nasal administration. Due to the lipophilic nature of the SC barrier, peptides existing in zwitterionic form can only permeate minimally through skin. To this end, molecular modification of peptide by increasing its lipophilicity may enhance its skin permeation. In a previous study, we have shown that enhanced skin permeation can be achieved by modifying the structure of a hexapeptide to make it more lipophilic.

Chemicals can interact with the intercellular lipids inside SC to enhance drug permeation through skin. Many CPEs, such as sulphoxides, fatty acids and terpenes, play an important role in promoting drug permeation through skin. It has been reported that the lipophilicity of the CPEs is proportional to their enhancing effects.

In addition to chemical enhancement, there are also physical methods, e.g., electroporation, iontophoresis, diamond microdermabrasion, laser radiation, and ultrasound. But disadvantages such as high costs have limited their application. Recently, MNs have emerged as a powerful tool to enhance the skin permeation of a variety of biomolecules, including oligonucleotides, peptides, proteins, and vaccines. MNs can be applied to the skin surface to create an array of microscopic passages through which the biomolecules can reach the dermis. Previously, we reported the application of microneedles on transdermal delivery of lidocaine, copper-peptide, and nucleic acids.

In this study, we investigated the skin permeation of decapeptide-12 (the native peptide, FIG. 1a) and its analogue (the palm-peptide, FIG. 1b) with higher lipophilicity, on their ability to permeate via skin using both CPEs and MNs. Propylene glycol (PG) is a widely used CPE and shows synergistic action when used together with other enhancers. Oleic acid, camphor, and menthol can increase drug permeation through skin. The efficacy of different peptide delivery systems was determined using human cadaveric skin samples.

TABLE 1
Physicochemical properties of the
peptides based on in silico prediction.
Physicochemical Properties

| | Native | Palm-D- |
|---|---|---|
| Properties | peptide | ISO-Amid |
| LogP | −6.462 | −0.751 |
| MW | 1395.54 | 1632.96 |
| H-donors | 19 | 18 |
| H-acceptors | 28 | 29 |
| Rot. Bonds | 52 | 66 |
| Rings | 5 | 5 |
| Solubility | 1000 mg/mL | 2.576 mg/mL |

Methods

Materials

Peptide and palm-peptide were synthesized by BIO BASIC (Ontario, Canada). PG and PBS tablets were purchased from VWR, USA. Oleic acid, penicillin-streptomycin, and trifluoroacetic acid were purchased from Sigma-Aldrich, USA. Camphor powder was purchased from New Directions, Australia. Menthol crystals was purchased from WFmed, USA. Phosphoric acid solution (85 weight percent in H2O) was purchased from SAFC, USA. Acetonitrile was obtained from RCI labscan, Thailand. Methanol was obtained from Honeywell, USA. Human epidermis was provided by Science Care, USA. All materials were used as supplied without further purification.

Peptide Synthesis and Characterization

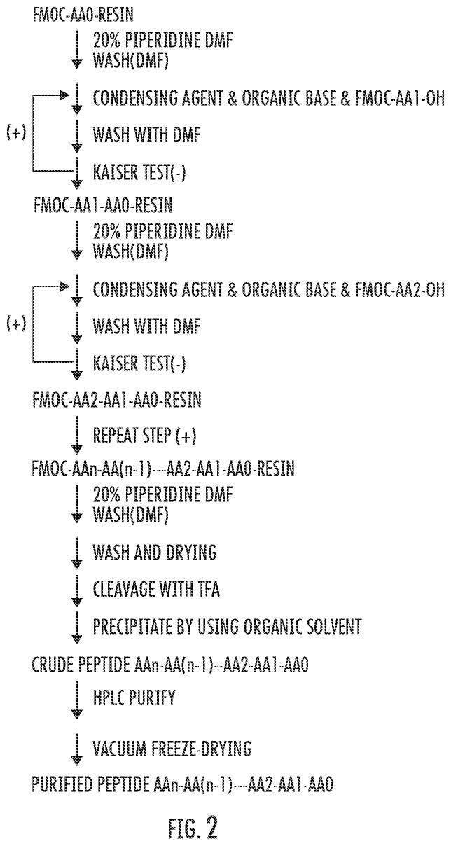

The peptide and analogue were synthesized through the conventional methods for solid phase chemical peptide synthesis, using FMOC based synthetic methodology on Rink-amide resin with modifications specific to each compound (FIG. 2). Purification of the final product were achieved by preparative high-performance liquid chromatography (HPLC).

Peptide Analogue Physicochemical Properties

In silico prediction of physicochemical properties of two peptides were performed.

Saturated Peptide Solution Preparation

The solubility of the various peptide analogues was determined in PG to prepare saturated peptide solutions. Excess peptide was added into each solvent inside a 2-millileter Eppendorf tube which was kept shaking in an incubation orbital shaker (OM15C, Ratek, Australia) for 48 hours. The entire set up was kept at room temperature. Samples were then centrifuged at 12,000 revolutions per minute (rpm) for 10 minutes. Subsequently, the supernatant was transferred into an amber colored ampoule bottle for assay.

Human Skin Membrane Preparation

Human dermatomed skin was obtained from Science Care (Arizona, USA). The skin tissues were excised from the thighs of two male cadavers with age at death of 66 and 57. The skin samples used in the study were without identifier and exempted from ethical review. The Integrity of cadaver skin was checked using visual inspection before use to ensure no pores or breaks in the skin surface were present. In addition, any compromise in skin integrity was identified through observation of a rapid and large increase in the amount of permeated peptide through the skin at the first sampling time point.

Fabrication of Micromould

Using a previously reported method, the molds were fabricated via thermal curing of Polydimethylsiloxane (PDMS) with embedded 3M Microchannel Skin System as the master template. Briefly, the elastomer and curing agent were mixed and vacuumed at 95 kilopascals for 10 minutes to 20 minutes to remove the entrapped air bubbles. Then, the mixture was poured slowly into the plastic petri dish. Later, it was kept for curing in hot air oven at 70 degrees Celsius for 2 hours. The cured PDMS was gently taken out from the petri dish using a surgical blade. The MN master was gently peeled off from the cured PDMS to get the PDMS mold.

Fabrication of Menthol-Based MN Patches Bearing Palm-Peptide

The PDMS molds were heated on a hot plate at 65 degrees Celsius for approximately 10 minutes. While heating the PDMS molds, palm-peptide was melted in cuvettes at 65 degrees Celsius using a heating block (Major Science) until all contents melted. Two percent palm-peptide was added into menthol and mixed well. PDMS molds were then removed from the hot plate and the mixed materials poured into the cavity. The molds were placed at room temperature and subsequently de-molded when the material solidified.

In Vitro Skin Permeation Testing

Franz type diffusion cells were used. Human epidermis was mounted between donor and receptor compartments and excessive skin at the sides was trimmed off to minimize lateral diffusion. SC was faced towards the donor compartment and the skin area for permeation was 1.327 square centimeters. The receptor compartment was filled with 5.5 milliliters of 1 millimolar PBS solution containing 1 percent (volume/volume) antibacterial antimycotic solution. Receptor solution was filtered twice to prevent formation of air bubbles beneath the epidermis. Two hundred and fifty microliters of solution was added to the donor compartment and covered with wraps. MN patches bearing peptide were first cut into the same proper size. Patches were then applied to the skin using a finger-thumb grip for 20 seconds. Scotch tape was used to fix the patches onto the skin. Triplicates were prepared. The donor compartment and the sampling port were covered with wraps to minimize gel contamination and evaporation. Magnetic stirrers were used at 180 revolutions per minute to mix the receptor solution during the study. The device was kept at 32 degrees Celsius. Five hundred microliters of the receptor fluid were collected at different time intervals for HPLC analysis and replaced with 500 microliters of fresh PBS containing 1 percent (volume/volume) antibacterial antimycotic solution. Permeation was measured over 24 hours.

HPLC Drug Analysis

The amount of peptide permeated was determined using Shimadzu CBM-20A HPLC system with Agilent ODS C18 column (4.6 millimeters x 250 millimeters x 5 microns, 170 Angstroms). The mobile phase consisted of mobile phase A (0.1 percent volume/volume trifluoroacetic acid in water) and mobile B (0.1 percent volume/volume trifluoroacetic acid in acetonitrile) with a gradient elution program with ratios of solvents A and B as follows: native peptide (15 percent to 35 percent B for 0 to 20 minutes and 15 percent for 20.01 minutes to 30 minutes), palm-peptide (55 percent to 45 percent B for 0 to 10 minutes and 55 percent for 10.01 minutes to 30 minutes) The flow rate was 1.0 milliliters per minute. The injection volume was 50 microliters and ultraviolet detection was performed at 280 nanometers. A calibration curve was established using the standard solutions from 5 micrograms per milliliter to 50 micrograms per milliliter for native peptide and from 1 micrograms per milliliter to 50 micrograms per milliliter for palm-peptide.

Statistical Analysis

All data were collated and prepared using GraphPad Prism 8 (GraphPad Software Inc, CA, USA). Statistical significances were calculated using ANOVA, with p less than (<) 0.05 considered statistically significant.

Results

Peptide Modification and Physicochemical Properties

The palm-peptide was modified from its parent compound, namely, the native peptide, to reduce the formation of zwitterions and increase lipophilicity (FIGS. 1a and 1b). For palm-peptide, N-terminal was palmitoylated, C-terminal was modified to amide, and the tyrosine at position 6 was changed from L- to D-. After C-terminal esterification, as seen in palm-peptide, the originally ionizable carboxyl ground no longer formed charged ions. The palm-peptide showed higher Log P of −0.751 than the native peptide. However, both peptides have high molecular weight, making it difficult for them to permeate through skin.

In Vitro Skin Permeation of Peptides

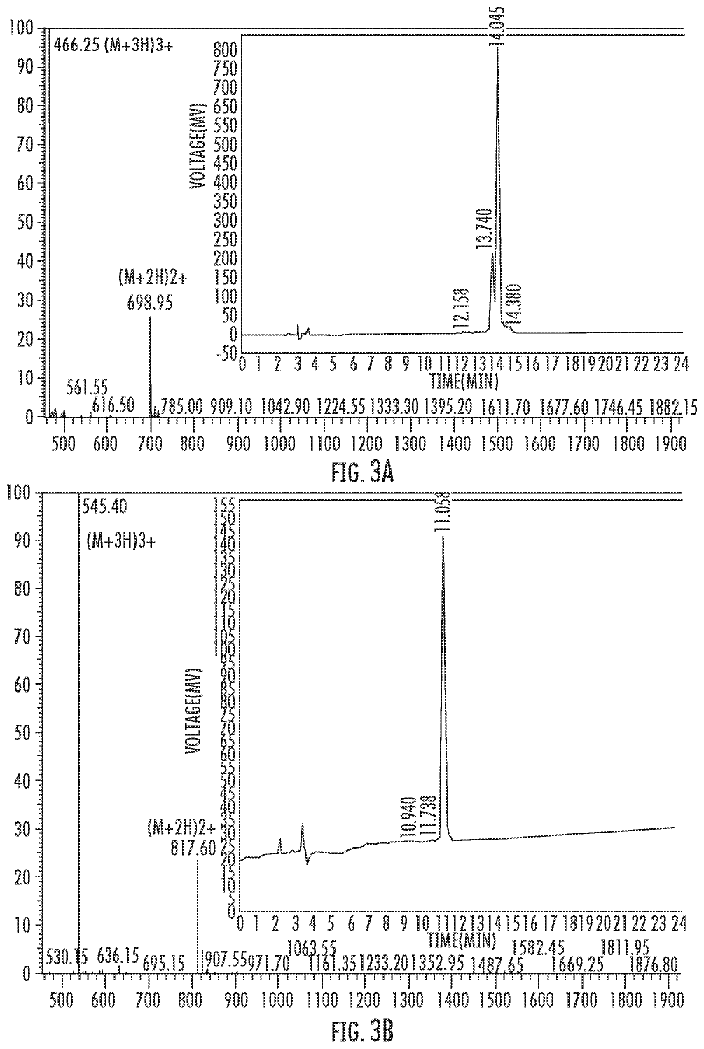

PG is commonly used in cosmetics, well known for its co-solvent and permeation enhancing effect. Hence, pure PG and PG containing 5 percent (weight/volume) enhancer were also used to prepare peptide solutions. The amount of native peptide in MN patch and palm-peptide in different drug delivery systems is shown in FIG. 3a. The highest permeation of palm-peptide was observed in PG containing 5 percent (weight/volume) menthol. After normalization against dose, as shown in FIG. 3b, a significant higher percentage of native peptide permeated through skin in the MN patch than in other formulations (P less than (<) 0.0001).

Cumulative Peptide Permeation in 24 Hours

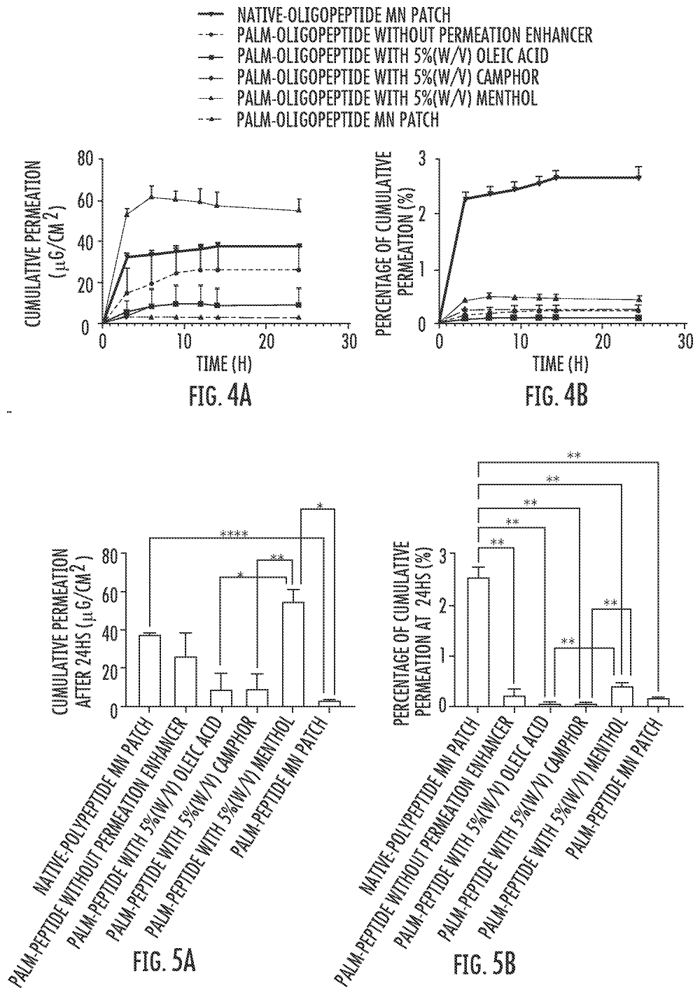

The cumulative permeation of the two peptides in 24 hours from different carriers is shown in FIGS. 4a and 4b. The highest permeation of palm-peptide was found in 5 percent (weight/volume) menthol (FIG. 4a). For MN patches, a significantly higher amount of native peptide permeated through skin than the palm-peptide (P less than (<) 0.0001). After normalization by dose (FIG. 4b), the native peptide permeation in MN patch was significantly higher than others (P less than (<) 0.01).

Peptide Human Skin Retention

Since the peptides reduce epidermal melanin content, we measured skin retention of the two peptides after 24 hours (FIGS. 5a and 5b). No peptides were detected in the skin samples for the following three formulations: native peptide in MN patch, palm-peptide in 5 percent camphor, and palm-peptide in 5 percent menthol. For the rest of the formulations, no significant differences were found among them (FIG. 5a). After dose normalization (FIG. 5b), the percentage of palm-peptide retained in skin, when using MN patch, was significantly higher than the other two formulations (P less than (<) 0.01, P less than (<) 0.001).

Discussion

The Skin Barrier and Modification of the Peptides

The outermost lipophilic layer of the skin, called the SC, is the major obstacle to percutaneous penetration. This lipophilic layer ensures that only small and moderately lipophilic molecules can traverse the skin in sufficient quantities to elicit therapeutically relevant effects. Peptides with large molecular weight and hydrophilic properties cannot penetrate SC in sufficient amounts. Therefore, we modified the termini of native peptide and synthesized a new peptide, namely, palm-peptide. Because of the higher lipophilicity of palm-peptide than native peptide, there was a corresponding increase in amount of permeation and skin retention in 24 hours (FIGS. 4a and 4b and FIGS. 5a and 5b) in PG solutions. It suggested the feasibility of chemical modification in promoting transdermal delivery of peptides using PG solution.

The Skin Retention of the Peptides

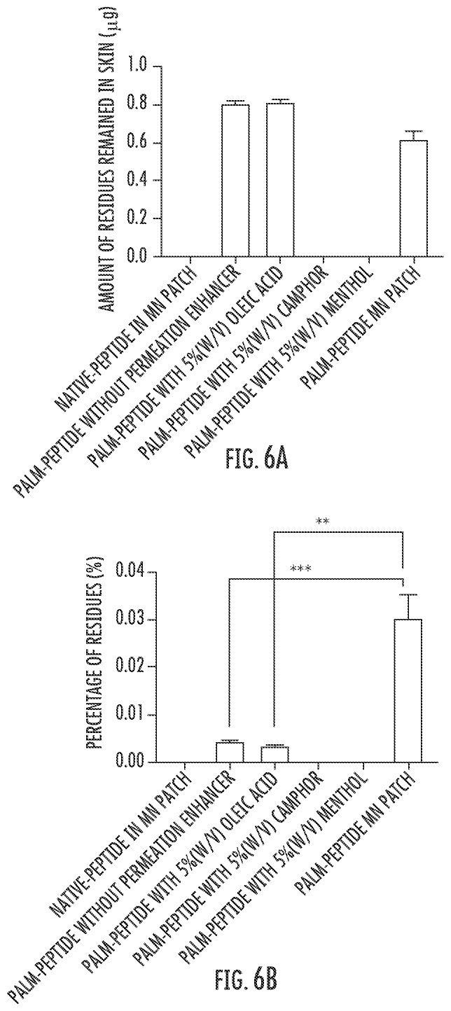

Since the two peptides target melanocytes to exert their therapeutic effect, and melanocytes are located in the basal layer of the epidermis, we assessed the retention of the peptides in side skin membranes. In comparison to undetectable skin retention of native peptide when delivered using MN patches, there was apparent skin retention of palm-peptide when delivered in MN patches (FIGS. 6a and 6b). Therefore, structural modification of native peptide can largely increase its efficacy topically by increasing its skin retention.

TABLE 2
The properties of three chemical penetration enhancers.
Physicochemical Properties

| Properties | Oleic acid | Camphor | Menthol |
|---|---|---|---|
| LogP | 7.421 ± 0.199 | 2.089 ± 0.300 | 3.216 ± 0.204 |
| MW | 282.46 | 152.23 | 156.26 |
| Solubility | 0.0023 mg/mL | 1.1 mg/mL | 1.5 mg/mL |

The Effect of CPEs on Skin Permeation of the Peptides

CPEs have been investigated to promote drug transdermal delivery for decades and many have improved transdermal delivery. One mechanism of action has been described as “pull-push” effect. High solubility parameter difference between CPE and the drug would push the drug molecules through SC. According to the theory and considering the property differences between the two peptides, we selected oleic acid with high lipophilicity and hydrophilic camphor and menthol (Table 2), which have previously been reported to enhance transdermal penetration. We expected PG containing 5 percent (weight/volume) oleic acid would exhibit the best improvement of skin permeation and skin retention due to the greatest Log P difference from both peptides.

However, results were not consistent with the expectation that oleic acid will have the best enhancing effect on skin permeation. For the native peptide, the three enhancers didn't alter skin permeation. For palm-peptide, camphor and oleic acid showed inhibitory effects, while menthol had an enhancing effect on skin permeation. This may be due to the interaction between CPEs and skin leading to altered partition behavior of palm-peptide.

The Effect of MNs on Skin Permeation of the Peptides

In this study, both peptides in MN patches showed more benefits than CPEs, in terms of skin permeation. Moreover, the native peptide showed better skin permeation than palm-peptide. It can be attributed to the higher solubility of native peptide in water than the palm-peptide. MNs destroyed the skin barrier by creating microscale passages through skin, so that native peptides in MN patches can directly transport into the receptor chamber. Native peptide, as a macromolecule with high polarity, tend to diffuse into the receptor PBS solution, in which native peptide is miscible.

The Effect of MNs on Skin Retention of Peptides

For skin retention, the highest percentage of palm-peptide retained inside skin was observed in MN patch, significantly different from other groups, although the percentage of palm-peptide in MN patch retention inside skin was still very low (less than (<) 0.1 percent). This may be because MNs destroyed the skin barrier, peptides directly transported into dermis and epidermis. Besides, higher lipophilicity of palm-peptide facilitated transcutaneous penetration. On the other hand, no native peptides were detected in the skin samples after 24 hours. This may be due to two reasons. First, the native peptide is more polar than the palm-peptide, leading to its diffusion into the aqueous solution in the receptor compartment. Second, the amount of peptide inside the MN patch is very limited resulting in minimal native peptide retention in skin despite high permeation.

Estimation of Palm-Peptide Concentration Inside Skin

The highest of palm-peptide concentration in skin reached 9.0 micrograms per gram, which was close to effective concentration of cell experiments (about 13.95 micrograms per gram, estimated from 10 micromolar); what's more, according to cytotoxic test, only when concentration exceeds 100 micromolar peptides inhibit melanocytes viability and proliferation, far exceeding the concentration that can be achieved in the skin. However, there was no evidence for the efficacy of palm-peptides concentration in skin and therefore, more detailed pharmacodynamic experiments need to be performed.

Infinite Dose Versus Finite Dose in the Donor Compartment

Depletion of permeants in the donor side over the course of the experiment usually results in a reduction in the rate of permeation and an eventual plateau in the cumulative permeation profile. On the other hand, if the permeant is applied as an infinite dose, there is sufficient permeants in the donor side to make any changes in donor concentration negligible. Infinite dose experiments are those with typical dose of more than 10 milligrams per square centimeter in the donor compartment.

In this study, MN patch was applied as finite dose condition and exhibited a decreasing rate and a plateau in cumulative permeation profile (FIGS. 3a and 3b). However, the theory was not applicable to the permeation profile of palm-peptide solutions, which was applied as infinite dosage condition (greater than (>) 13 milligrams per square centimeter) yet resulted in a rate reduction as well.

Ideal Log P for Transdermal Delivery and Peptide Modification

Previous studies have shown that the ideal Log P for drug penetration is about 2. This represents a large difference between the Log P of palm-peptide (−0.751) and the ideal value.

In this study, we modified the native peptide to increase its lipophilicity. Previously, we reported the improvement of peptides penetration across skin by changing functional groups of side chains, which increased lipophilicity from −6.37 of Log P to 1.75. That may represent a feasible solution.

### CONCLUSION

In study, we used chemical modification, CPEs and MN patch to improve the skin permeation and retention of peptides. CPEs exhibited a positive effect on skin permeation but did not promote skin retention of palm-peptide. MN patches improved both skin permeation and retention of palm-peptide. For the peptides to target melanocytes located in the epidermal basal layer, structural modification and MN patches could improve their efficacy. To further improve skin retention of peptide, modifying the structure of amino side chains rather than terminals may be tested.

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

1: A method of treating a subject by performing permeation of a cell, the method comprising:
administering to a subject in need thereof a composition comprising an effective amount of one or more peptides, wherein the one or more peptides comprise:

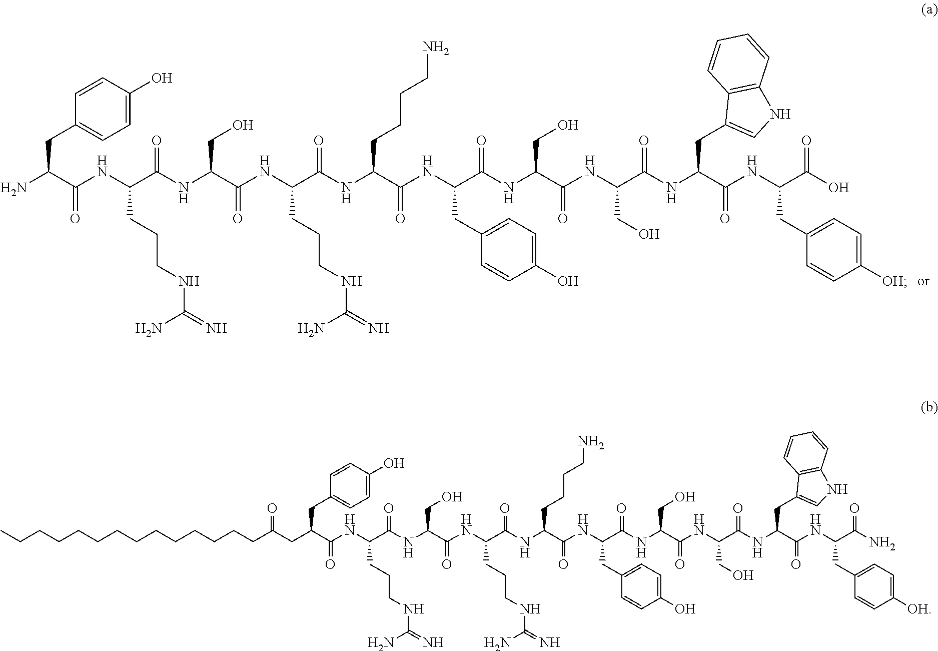 [SMILES: N=C(N)NCCC[C@H](NC(=O)[C@H](CO)NC(=O)[C@H](CCCNC(=N)N)NC(=O)[C@@H](N)Cc1ccc(O)cc1)C(=O)N[C@@H](CCCCN)C(=O)N[C@@H](Cc1ccc(O)cc1)C(=O)N[C@@H](CO)C(=O)N[C@@H](CO)C(=O)N[C@@H](Cc1c[nH]c2ccccc12)C(=O)N[C@@H](Cc1ccc(O)cc1)C(=O)O ; CCCCCCCCCCCCCCC(=O)C[C@@H](Cc1ccc(O)cc1)C(=O)N[C@@H](CCCNC(=N)N)C(=O)N[C@@H](CO)C(=O)N[C@@H](CCCNC(=N)N)C(=O)N[C@@H](CCCCN)C(=O)N[C@@H](Cc1ccc(O)cc1)C(=O)N[C@@H](CO)C(=O)N[C@@H](CO)C(=O)N[C@@H](Cc1c[nH]c2ccccc12)C(=O)N[C@@H](Cc1ccc(O)cc1)C(N)=O]

2: The method according to claim 1, wherein the cell is a mammalian cell.

3: The method according to claim 2, wherein the mammalian cell is a skin cell.

4: The method according to claim 3, wherein the skin cell is a melanocyte.

5: The method according to claim 1, wherein the administration is by a microneedle.

6: The method according to claim 1, wherein the administration is by a chemical penetration enhancer.

7: The method according to claim 6, wherein the chemical penetration enhancer comprises at least one of propylene glycol, Oleic acid, camphor, and menthol.

8: The method according to claim 7, wherein the one or more peptides are present with menthol in a concentration of about 5 percent (weight/volume).

9: The method according to claim 1, wherein
the one or more peptides consist of 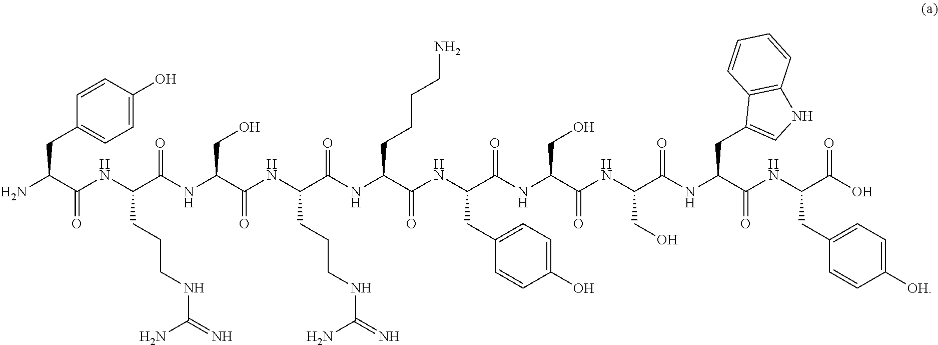 [SMILES: N=C(N)NCCC[C@H](NC(=O)[C@H](CO)NC(=O)[C@H](CCCNC(=N)N)NC(=O)[C@@H](N)Cc1ccc(O)cc1)C(=O)N[C@@H](CCCCN)C(=O)N[C@@H](Cc1ccc(O)cc1)C(=O)N[C@@H](CO)C(=O)N[C@@H](CO)C(=O)N[C@@H](Cc1c[nH]c2ccccc12)C(=O)N[C@@H](Cc1ccc(O)cc1)C(=O)O]
and the administration is by microneedle.

10: The method according to claim 1, wherein
the one or more peptides consist of 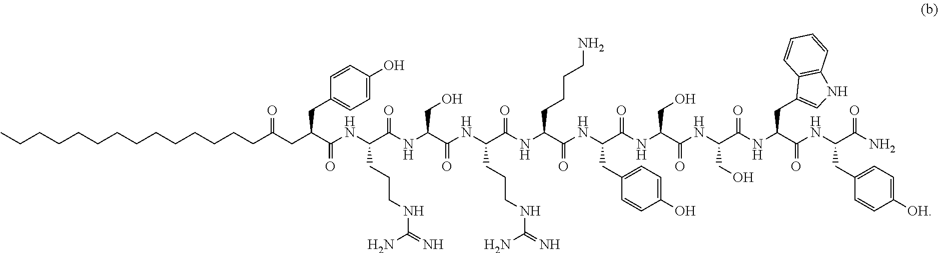 [SMILES: CCCCCCCCCCCCCCC(=O)C[C@@H](Cc1ccc(O)cc1)C(=O)N[C@@H](CCCNC(=N)N)C(=O)N[C@@H](CO)C(=O)N[C@@H](CCCNC(=N)N)C(=O)N[C@@H](CCCCN)C(=O)N[C@@H](Cc1ccc(O)cc1)C(=O)N[C@@H](CO)C(=O)N[C@@H](CO)C(=O)N[C@@H](Cc1c[nH]c2ccccc12)C(=O)N[C@@H](Cc1ccc(O)cc1)C(N)=O]
and the administration is by chemical penetration enhancer.

11: A peptide consisting of:

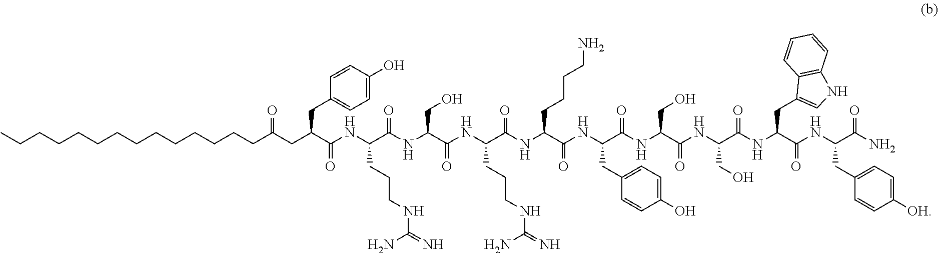 [SMILES: CCCCCCCCCCCCCCC(=O)C[C@@H](Cc1ccc(O)cc1)C(=O)N[C@@H](CCCNC(=N)N)C(=O)N[C@@H](CO)C(=O)N[C@@H](CCCNC(=N)N)C(=O)N[C@@H](CCCCN)C(=O)N[C@@H](Cc1ccc(O)cc1)C(=O)N[C@@H](CO)C(=O)N[C@@H](CO)C(=O)N[C@@H](Cc1c[nH]c2ccccc12)C(=O)N[C@@H](Cc1ccc(O)cc1)C(N)=O]

12: A therapeutic agent for treating a subject with a condition comprising: an effective amount of one or more peptides, wherein the one or more peptides comprise:

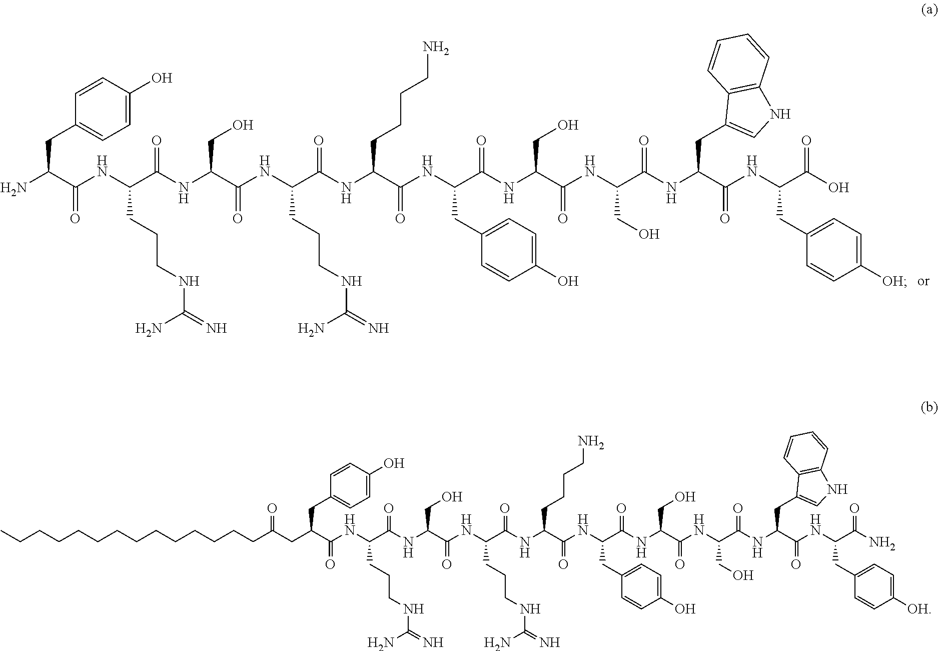 [SMILES: N=C(N)NCCC[C@H](NC(=O)[C@H](CO)NC(=O)[C@H](CCCNC(=N)N)NC(=O)[C@@H](N)Cc1ccc(O)cc1)C(=O)N[C@@H](CCCCN)C(=O)N[C@@H](Cc1ccc(O)cc1)C(=O)N[C@@H](CO)C(=O)N[C@@H](CO)C(=O)N[C@@H](Cc1c[nH]c2ccccc12)C(=O)N[C@@H](Cc1ccc(O)cc1)C(=O)O ; CCCCCCCCCCCCCCC(=O)C[C@@H](Cc1ccc(O)cc1)C(=O)N[C@@H](CCCNC(=N)N)C(=O)N[C@@H](CO)C(=O)N[C@@H](CCCNC(=N)N)C(=O)N[C@@H](CCCCN)C(=O)N[C@@H](Cc1ccc(O)cc1)C(=O)N[C@@H](CO)C(=O)N[C@@H](CO)C(=O)N[C@@H](Cc1c[nH]c2ccccc12)C(=O)N[C@@H](Cc1ccc(O)cc1)C(N)=O]

13: The therapeutic agent according to claim 12, wherein the condition comprises at least one of hyperpigmentation, melasma, neurological, dermatological, oncological, immunological, anti-inflammatory functions, anti-senescence functions, integumentary system, skeletal system, muscular system, lymphatic system, respiratory system, digestive system, nervous system, endocrine system, cardiovascular system, urinary system, reproductive systems, musculoskeletal system, organs of special sense (optical), auditory, infectious diseases, immune disorders, nutritional deficiencies, respiratory system, cardiovascular system, digestive system, genitourinary system, hemic and lymphatic system, skin, endocrine system, neurological conditions, mental disorders, or dental and oral.

14: The therapeutic agent according to claim 12, further comprising a chemical penetration enhancer.

15: The therapeutic agent according to claim 14, wherein the chemical penetration enhancer comprises at least one of propylene glycol, Oleic acid, camphor, and menthol.

16: The therapeutic agent according to claim 14, wherein:
the chemical penetration enhancer comprises menthol; and
the at least one or more peptides consist of

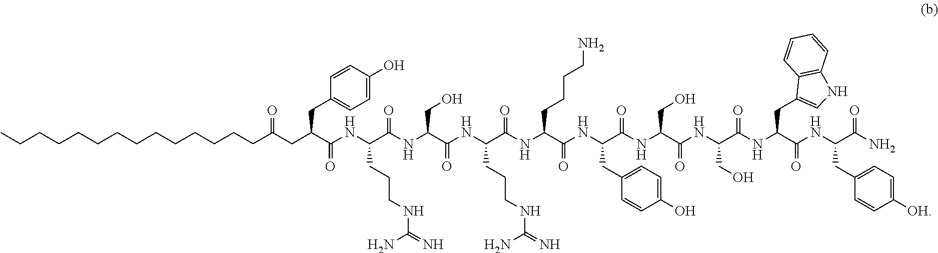 [SMILES: CCCCCCCCCCCCCCC(=O)C[C@@H](Cc1ccc(O)cc1)C(=O)N[C@@H](CCCNC(=N)N)C(=O)N[C@@H](CO)C(=O)N[C@@H](CCCNC(=N)N)C(=O)N[C@@H](CCCCN)C(=O)N[C@@H](Cc1ccc(O)cc1)C(=O)N[C@@H](CO)C(=O)N[C@@H](CO)C(=O)N[C@@H](Cc1c[nH]c2ccccc12)C(=O)N[C@@H](Cc1ccc(O)cc1)C(N)=O]

17: The therapeutic agent according to claim 16, wherein the menthol is present at about 5 percent by weight.

18: A kit for treating a condition comprising the therapeutic agent according to claim 12.

19: The kit according to claim 18, further comprising a microneedle.

20: The kit according to claim 18 further comprising a chemical penetration enhancer.
